(19) United States
(12) Patent Application Publication
(10) Pub. No.: US 2005/0273177 A1
(43) Pub. Date: Dec. 8, 2005

(54) Prosthetic device having drug delivery properties

(76) Inventors: David P. Summers, Montgomery, TX (US); Diane Dottavio, Houston, TX (US)

Correspondence Address: NICK A NICHOLS, P O BOX 16399, SUGARLAND, TX, 774966399, US

(21) Appl. No.: 10/862,901

(22) Filed: Jun. 7, 2004

(51) Int. Cl. A61F 2/04; A61F 2/06

(52) U.S. Cl. 623/23.7; 623/1.42; 424/424

(57) ABSTRACT

A prosthesis includes suitable mechanical properties needed to open and maintain a vessel, duct, tract, or organ in a mammalian body. The prosthesis includes a pharmaceutical coating containing a pharmaceutical agent. The pharmaceutical agent is capable of acting upon and altering the mechanisms of biologic systems in a manner providing a medicinal therapy. The prosthesis includes at least one layer of the pharmaceutical coating overlaid by a permiable membrane.

### BACKGROUND OF THE DISCLOSURE

The present invention relates generally to the field of implantable medical devices, more specifically to a method of localized drug delivery from a prosthetic device having a drug or pharmaceutical material coated thereon and adapted to be placed withing a blood vessel, duct, tract or organ of a mammalian body.

The use of implantable medical devices to treat a variety of medical conditions by introducing the devices into a body cavity, tract, duct or vessel has become common medical practice. Treatment of blood vascular disease such as occlusions, obstructions, and stenosis of the blood vessels resulting from atherosclerosis, a disease of atherosclerotic plaques and cholesterol deposits, routinely employ the use of small metal scaffolds called intravascular stents to ameliorate the ischemic condition caused by these blockages. The use of stents is described in greater detail in U.S. Pat. No. 5,824,649, and more particularly in U.S. Pat. No. 5,980,551 which incorporates the use of a biodegradable substrate which is loaded with a drug or active agent to be chronically released within a mammalian body. The invention briefly described above sought to accomplish the opening and maintenance of the opening of a blood vessel by mechanical means while providing medicinal drug treatment from the gradual release of drugs from a slowly degrading biocompatible substrate coating on the intravascular stent. Such stents are capable of chronic release of various drugs for a period of a few days to a period of many months.

Depositing a drug or pharmaceutical agent onto a metal surface, such as a stent, and determining the release rate of the drug before it is used is a difficult problem encountered by prior art devices. All drugs and pharmaceutical agents have different levels of therapy and toxicity which should be determined before the drug is administered to a patient. It would be desirable therefore to more precisely control the chronicity of the drug treatment over short term regimens (hours and days) or longer term (weeks and months). It would also be desirable that the delivery of medication be subject to precise control, and systemic exposure to the medication limited. This would be particularly advantageous in therapies, such as chemotherapy, which requires the delivery of chemotherapeutic agents to a particular organ or treatment site.

It is therefore an object of the present invention to provide a prosthetic device to deliver a pharmaceutical agent to diseased organs, blood vessels, tissues, systems, circuits, or networks (such as neural networks). This is accomplished by coating the prosthetic device with a pharmaceutical agent and placing it directly into such organs, tissues, systems, circuit or networks, or proximal to such sites (e.g., a feeding artery of a tumor) and controlling the release of the pharmaceutical agent from the prosthetic device over time.

It is another object of the present invention to provide a prosthetic device having a pharmaceutical coating thereon overlaid by a permiable membrane.

It is another object of the present invention to provide a prosthetic device for controlling the osmotic release of a drug or pharmaceutical agent within a mammalian body.

It is another object of the present invention to provide a stent coated with a pharmaceutical agent and covered with a permiable membrane.

It is another object of the present invention to provide a prosthetic device having a biodegradable polymer substrate coated with a therapeutic drug overlaid with a non-erodable permiable membrane.

It is another object of the present invention to provide a prosthetic device having a biodegradable polymer substrate with a therapeutic drug incorporated in the polymeric material.

### SUMMARY OF THE INVENTION

The present invention comprises a prosthetic device having suitable mechanical properties needed to open and maintain a vessel, duct, tract, or organ and controlling the release and delivery of a pharmaceutical agent carried on the prosthetic device. The pharmaceutical agent is capable of acting upon and altering the mechanisms of biologic systems in a manner providing a medicinal therapy. The prosthetic device includes at least one layer or coating of the pharmaceutical agent applied thereon and overlaid by a permiable membrane for controlling the osmotic release of the pharmaceutical agent into the vessel, duct, tract or organ over time.

### BRIEF DESCRIPTION OF THE DRAWINGS

So that the manner in which the above recited features, advantages and objects of the present invention are attained can be understood in detail, a more particular description of the invention briefly summarized above, may be had by reference to the embodiments thereof which are illustrated in the appended drawings.

It is noted, however, that the appended drawings illustrate only typical embodiments of this invention and are therefore not to be considered limiting of its scope, for the invention may admit to other equally effective embodiments.

FIG. 1A is a partial side view of a prosthetic device located within a blood vessel;

FIG. 1B is a section view of a segment of a prosthetic device coated with a pharmaceutical agent in accordance with the invention;

FIG. 2A illustrates the release profile of a pharmaceutical preparation containing 100 μg PGE-1 applied on a prosthetic device and overlaid with a permiable membrane;

FIG. 2B illustrates the release profile of a pharmaceutical preparation containing 250 μg PGE-1 applied on a prosthetic device and overlaid with a permiable membrane;

FIG. 3A illustrates the release profile of a pharmaceutical preparation containing 100 μg PGE-1 applied on a prosthetic device and overlaid with a permiable membrane having greater thickness than the membrane of FIG. 2A; and

FIG. 3B illustrates the release profile of a pharmaceutical preparation containing 250 μg PGE-1 applied on a prosthetic device and overlaid with a permiable membrane having greater thickness than the membrane of FIG. 2B.

### DETAILED DESCRIPTION OF A PREFERRED EMBODIMENT

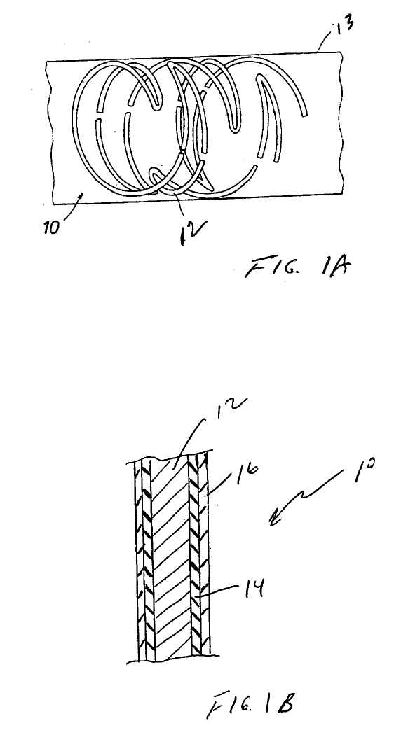

In a preferred embodiment, the prosthetic device of the invention is generally identified in FIGS. 1A and 1B by the reference numeral 10. The prosthetic device 10 comprises a base structure 12 adapted for introduction into a mammalian body. By way of example, the base structure 12 of the invention may be configured into a vascular stent particularly suitable for insertion into the vascular system of a human patient, such as a blood vessel 13. The stent 12 is coated with a pharmaceutical mixture or coating containing a drug or pharmaceutical agent which is to be released over time for treating a medical condition. The pharmaceutical mixture applied on the stent 12 forms a coating or layer 14 thereon. The coating layer 14 is overlaid by a permiable membrane 16. The permiable membrane 16 encapsulates the layer 14 about the stent 12. The stent 12 and permiable membrane 16 form an osmotic pump which operates as interstitial fluid (from ducts, tracts or organs) or blood plasma fluid (from the circulatory system) is attracted through the membrane 16 to hydrate the pharmaceutical agents in the layer 14. As the pharmaceutical agents hydrate and drug hydrolysis is activated, the pressure gradient across the membrane 16 increases. An increase in the pressure gradient produces an egress of fluids through the permiable membrane 16 thereby expelling the drug from the pharmaceutical layer 14 on the surface of the stent 12 into the environment, such as a blood vessel, duct, tract or organ of the human patient. Alternatively, the loss of fluid through the membrane 16 may be recompensated by external pressure gradients which oscillate with pressure variations in the blood pressure or system itself.

In a preferred embodiment of the present invention, the pharmaceutical agent contained in the layer or coating 14 deposited on the stent device 12 may, for example, be prostaglandin E-1 (PGE-1), a naturally occurring fatty acid of the cyclopentenone family. The timed release of PGE-1 produces powerful chronic antagonistic chemotaxis to thromboxane and leukotrience actions on the platelets and injured vessel wall while modulating the proliferation of smooth muscle cells (SMC) and extracellular matrix within the media of the blood vessel, duct or the like. This two-stage process continues to produce inhibition of protein absorption and hence cellular interactions at the bio-material surface while releasing powerful inhibitions of platelet aggrandizement and modulators of cell growth in the region of the vessel where the stent is located. The protein inhibiting action of the biologically active agent continues over a predetermined period of hours, days, weeks or months or until endothelialization of the bio-surface is complete. These surfaces may be modified to serve as attachment sites for suitable bio-specific peptides that result in a surface that could potentially adhere to only one particular cell type, such as endothelial cells in the case of stent or vascular grafts.

In accordance with the present invention, anticancer, antiproliferative, preoperative tumor debulkers or chemotherapeutic agents may be delivered directly to a tumor. It should also be noted that pallatives which ease the symptoms of the disease such as anesthetics, analgesics, neural stimulators, agonists and antagonist are also included within the scope of the invention.

In the case of pallatives, for example, procaine or morphine may be administered for pain control, nicotine or nicotine receptor agonist may be placed in the vascular supply of the thalamic substantia nigra for treatment of neurodegenerative disease such as Alzheimer's, Parkinson's, Huntington's and Lou Gehrig's disease.

Immunosuppressive agents such as cyclosporin may be used in accordance with the invention to provide long-term immunosuppressive therapies. In the case of organ or tissue transplant therapies, hormones such as testosterone, estrogen use for steroid deficiencies, dexamethasone and various prostaglandins for inflammatory therapies are also contemplated for use in the instant invention.

The present invention is further described in the following Examples. However, those of ordinary skill in the art will readily determine that these examples are merely illustrative of the invention as defined in the claims which follow thereafter.

### EXAMPLES

### Example 1

Preparation of Stents Coated with PGE-1

Various materials presently used in pharmaceutical preparations as solubilizers, crystallization inhibitors, suspension stabilizers and lyophilization agents were mixed with PGE-1. The mixtures were dissolved in solvent systems composed of ethanol, chloroform, water, or mixtures thereof. Prior to coating, stents (provided by different stent manufacturers) were cleaned by sonification in absolute ethanol for thirty minutes, and dried. Each stent was weighed before and after coating. The amount of PGE-1 in the coating solution was determined by high performance liquid chromatography analysis (HPLC), and used to calculate the percent by weight of PGE-1 on the stent. To confirm this value, the coating was dissolved (from the stent) and the PGE-1 content analyzed by HPLC.

Drug coatings on the stents were assessed using scanning electron microscopy (SEM) for: (a) smoothness, (b) uniformity, and (c) lack of cracking. Stents that met these criteria, were overlaid with a thin membrane of non-erodable polymer matrix to retard the release of PGE-1 from the surface of the stents. Vapor-deposition of the polymer matrix was carried out according to the manufacturer's protocol, and the thickness of the polymer matrix membrane was measured using a Tencor device.

Using the most desirable coating combination, twenty coronary stents were coated with either 100 micrograms or 250 micrograms of PGE-1, and overlaid with the polymer matrix membrane. Five stents from each drug concentration were expanded by insertion of a balloon catheter. The stents were examined by SEM after deposition of the pharmaceutical layer 14, after applying the membrane 16, and after expansion of the stents 12.

PGE-1 elution profiles were determined for each group of stents 12. In vitro PGE-1 release curves were performed using HPLC. Drug elution profiles were measured in phosphate buffered saline (pH 7.4) using a LabQuake 360° degree rotator, at ambient temperature. At each time point, the stents were removed from the one milliliter sample (typically, after 24 hours) and transferred to the next aliquot. HPLC analysis of the eluent was performed using a Waters Alliance system equipped with a 996 photodiode array detector.

### HPLC Analysis of PGE-1 Eluted from Stents

HPLC Program Conditions/Settings:

Column: 4.6 mm×150 mm Reverse Phase C₁₈

Photo-diode array UV detector set to scan 190 to 300 nm

Column temp: 40° C.

Solvent system: 35% Acetonitrile/65% water with 0.1% acetic acid

Program: Isocratic

Flow rate: 1 ml/min

Time of run: 25 minutes

Sample injection: 100 μl

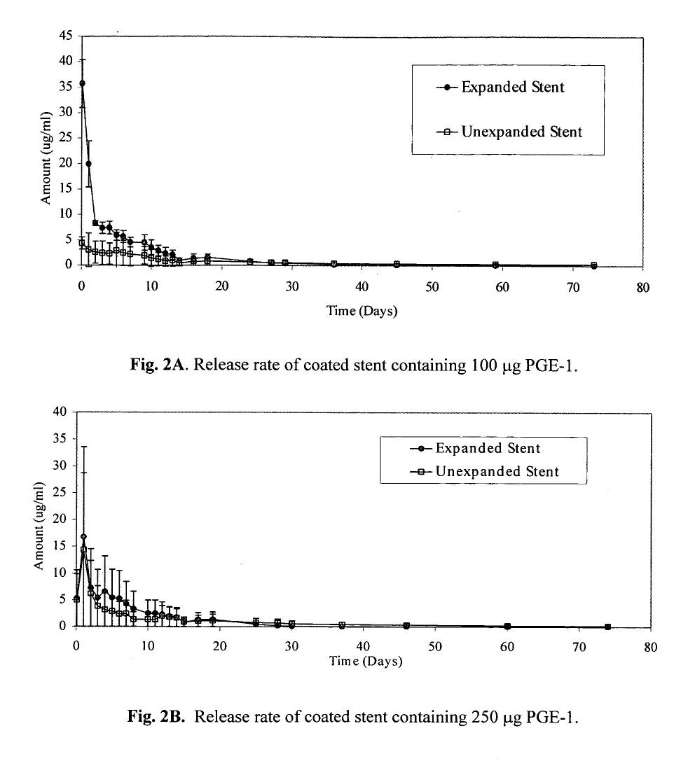

As seen in FIGS. 2A and 2B, the PGE-1 release profile is characterized by an initial “burst” of PGE-1 released during the first 48-96 hour period. The amount released and duration of the burst is dependent on the amount of PGE-1 coated on the stents. Routinely, the amount of PGE-1 released from the expanded stents was greater than from the unexpanded stents.

### Example 2

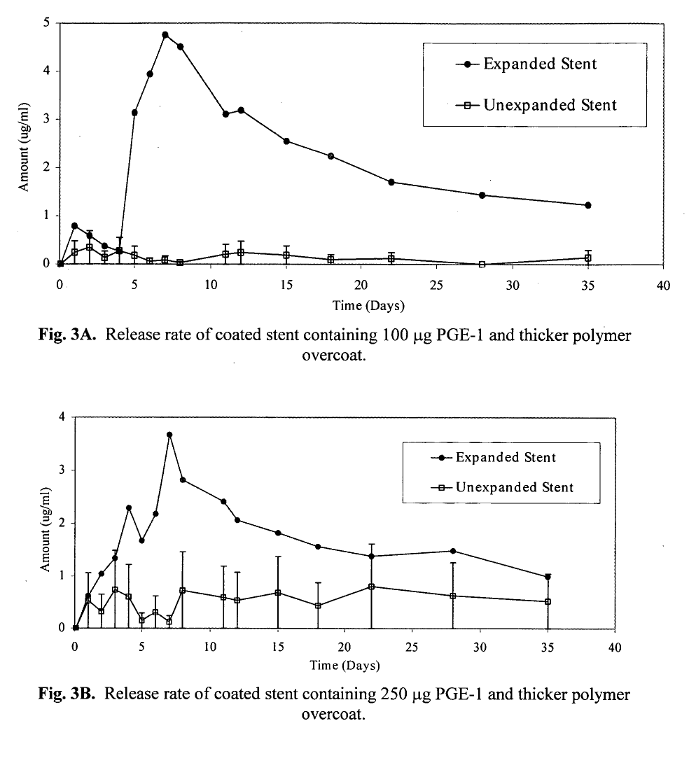

In Example 2 a second set of twenty stents were coated as described above, except the polymer membrane overcoat 16 was twice the thickness as the former. The release rates of PGE-1 from stents coated with the thicker polymer overcoat 16 are shown in FIGS. 3A and 3B. It will be noted that the initial release of PGE-1 is delayed and the overall amount of PGE-1 eluted from the stents 12 over time is reduced, due to the increased thickness of the polymer membrane 16.

TABLE 1
Total Recovery of PGE-1 from Coated Stents

| | Days | Study | Unxpanded | Expanded | Unxpanded | Expanded |
|---|---|---|---|---|---|---|
| PGE-1 Coated (μg) | | | 100 | 100 | 250 | 250 |
| PGE-1 Released (%) | 73 | 1 | 37% | 82% | 46% | 83% |
| PGE-1 Released (%) | 35 | 2 | 19% | 71% | 26% | 26% |

The experiment was designed to determine PGE-1 elution profiles of the coated stents. In FIGS. 2A and 2B, the release profiles are characterized by an initial “burst” of PGE-1 in the first 48-96 hours, followed by a slow, sustained release of the drug, which persists out to approximately sixty days. The amount of drug released and duration of the burst is dependent on (1) the amount of PGE-1 coated on the stent 12, and (2) the thickness of the permiable membrane 16. Typically, greater recovery of the drug is obtained from expanded stents compared to unexpanded stents.

In FIGS. 3A and 3B, the thickness of the membrane 16 was increased two-fold, as compared to the membrane 16 applied to stents in FIGS. 1A and 1B. This increase in membrane thickness results in a delayed release, and a decrease in the size of the initial “burst” of drug from the stents. Total recoveries of PGE-1 obtained from the coated stents are given in Table 1. Table 1 summarizes the average amount of PGE-1 recovered from the coated stents over the period of time indicated. Recoveries were higher from the stents coated with the thinner membrane 16.

While a preferred embodiment of the invention has been shown, and described, other and further embodiments of the invention may be devised without departing from the basic scope thereof, and the scope thereof is determined by the claims which follow.

1. A method of releasing a pharmaceutical agent into a mammalian body, comprising the steps of:
a) placing a prosthesis in said body;
b) said prosthesis including a coating applied thereon, said coating containing said pharmaceutical agent; and
c) releasing said pharmaceutical agent into said body.

2. The method of claim 1 including the steps of:
a) overlaying a permiable membrane over said coating; and
b) creating a pressure gradient across said permiable membrane for releasing said pharmaceutical agent into said body by osmotic action.

3. The method of claim 2 wherein said osmotic action results from hydrolysis of said pharmaceutical agent producing an oscillating pressure differential across said permiable membrane.

4. The method of claim 3 wherein said pharmaceutical agent is prostaglandin E-1.

5. The method of claim 2 including the step of controlling the rate of release of said pharmaceutical agent by increasing the thickness of said permiable membrane.

6. A prosthetic device for releasing a pharmaceutical agent within a mammalian body, comprising:
a) a prosthetic structure having at least one layer of a pharmaceutical mixture containing said pharmaceutical agent deposited on said structure; and
b) a permiable membrane overlaid over said at least one layer of pharmaceutical mixture.

7. The device of claim 6 wherein said prosthetic structure is a vascular stent.

8. The device of claim 7 wherein said pharmaceutical agent is prostaglandin E-1.

9. A stent coated with at least one layer of a pharmaceutical preparation for therapeutic treatment of a blood vessel, tract, duct or organ, said pharmaceutical preparation containing a pharmaceutical agent, and wherein said stent includes a non-erodable polymer membrane encapsulating said stent for retarding release of said pharmaceutical agent from said stent.

10. The stent of claim 8 wherein said pharmaceutical agent is prostaglandin E-1.
